(19) United States
(12) Patent Application Publication
(10) Pub. No.: US 2026/0158018 A1
(43) Pub. Date: Jun. 11, 2026

(54) PERFUSATE CONTAINING NICOTINAMIDE FOR INTRAOCULAR SURGERY, METHOD OF PREPARATION AND USE THEREOF

(71) Applicant: EYE INSTITUTE OF SHANDONG FIRST MEDICAL UNIVERSITY (SHANDONG EYE INSTITUTE, QINGDAO EYE HOSPITAL OF, Qingdao (CN)

(72) Inventors: Lixin XIE, Qingdao (CN); Xiaomin LIU, Qingdao (CN); Zongyi LI, Qingdao (CN); Xiaoming WU, Qingdao (CN); Qingjun ZHOU, Qingdao (CN); Ping LIN, Qingdao (CN); Haoyun DUAN, Qingdao (CN)

(73) Assignee: EYE INSTITUTE OF SHANDONG FIRST MEDICAL UNIVERSITY (SHANDONG EYE INSTITUTE, QINGDAO EYE HOSPITAL OF, Qingdao (CN)

(21) Appl. No.: 18/691,132

(22) PCT Filed: Nov. 15, 2023
(86) PCT No.: PCT/CN2023/131796

§ 371 Date: Mar. 12, 2024

(30) Foreign Application Priority Data
Oct. 26, 2023 (CN) 202311396074.7

(51) Int. Cl. A61K 31/455; A61K 9/00; A61K 9/08; A61P 27/02

(52) U.S. Cl. CPC A61K 31/455; A61K 9/0048; A61K 9/08; A61P 27/02

(57) ABSTRACT

The present disclosure belongs to the technical field of drug preparation and relates to a perfusate containing nicotinamide (NAM) for intraocular surgery, a method of preparation and use thereof. Provided is use of NAM in perfusates for intraocular surgery. In the present disclosure, the perfusate for intraocular surgery improved with NAM may inhibit corneal endothelial cell damage caused by phacoemulsification, reduce corneal edema caused by phacoemulsification, avoid a significant decrease in the number of corneal endothelial cells (CECs) caused by phacoemulsification, promote recovery of corneal transparency after phacoemulsification, promote recovery of the normal corneal thickness, maintain the normal density and cellular morphology of CECs, and regular expression of functional proteins, and reduce complications caused by phacoemulsification. The present disclosure may provide an effective strategy for realizing the rapid recovery of normal visual function of cataract patients after surgery.

This patent application is a national phase application of PCT application No. PCT/CN2023/131796 filed on Nov. 15, 2023, which claims the claims the benefit and priority of Chinese Patent application No. 202311396074.7 filed with the China National Intellectual Property Administration on Oct. 26, 2023, both of which are incorporated by reference herein in its entirety.

### TECHNICAL FIELD

The present disclosure belongs to the technical field of drug preparation, and specifically relates to a perfusate containing nicotinamide for intraocular surgery, a method of preparation and use thereof.

### BACKGROUND

Cataract ranks first of blinding eye disease worldwide. Currently, most cataract surgeries are performed by phacoemulsification, which uses high-intensity ultrasound energy to break up and emulsify the cloudy lens. With advances in surgical equipment and technological developments, the indications for phacoemulsification have been expanded to include mature cataracts with rigid lens nuclei, thus requiring greater ultrasonic emulsification energy and time. However, strong emulsification energy, fragmentation of the lens nucleus, and increased local temperature can damage corneal endothelial cells (CECs), causing a significant decrease in the number of CECs, which can lead to corneal edema, or even corneal endothelial decompensation. Corneal transplantation is required for corneal blindness patients. CECs are located in the innermost layer of the cornea and maintain normal corneal transparency and visual function through barrier and pump functions. Since the proliferation of adult CECs is extremely limited, they can only be repaired by peripheral cell expansion and migration after injury, resulting in a decrease in cell density over the years. When the cell density falls below the threshold (400-500 cells/mm²), corneal edema, decreased transparency, and in severe cases, corneal blindness can occur. Corneal endothelial decompensation such as corneal endothelial folds and corneal edema caused by phacoemulsification is more common, which seriously affects the recovery of patients'visual function. Previous studies have found that corneal endothelial decompensation caused by cataract surgery is the most common cause of penetrating keratoplasty. Cataract surgery has also become the leading indication for corneal endothelial transplantation, ranking second only to Fuchs endothelial dystrophy. As of now, there is no proven, convenient, feasible, and widely disseminated clinical treatment strategy to protect CECs during and after phacoemulsification.

It has been reported that corneal endothelial damage is partly caused by oxidative stress during the process of ultrasonic emulsification. Hydrogen (H₂) , as a strong reducing agent, can reduce corneal endothelial damage during ultrasonic emulsification when dissolved in the perfusate, but H₂ is highly penetrating, volatile, and difficult to be retained for a long period of time in the perfusate. Currently, the perfusate BSS PLUS promoted by Alcon Laboratories, Inc. shows protection of the corneal endothelium, but it is expensive. There is still a lack of effective and low-cost products to reduce corneal damage during and after phacoemulsification.

### SUMMARY

An objective of the present disclosure is to provide a perfusate containing nicotinamide (NAM) for intraocular surgery and a method of preparation and use thereof. The novel perfusate for intraocular surgery obtained by improved optimization using NAM can be applied to phacoemulsification to protect CECs with good effect and low cost.

The present disclosure provides use of NAM in the preparation of a perfusate for intraocular surgery.

In some embodiments, the intraocular surgery includes phacoemulsification for cataract and/or vitrectomy.

In some embodiments, the cataract is selected from the group consisting of senile cataract, diabetic cataract, cataract in period of compensation of corneal endothelial function, and cataract with corneal endothelial decompensation.

The present disclosure also provides a perfusate containing NAM for intraocular surgery, the perfusate including NAM and a balanced salt solution.

In some embodiments, NAM in the perfusate has a concentration of 1 to 5 mM.

In some embodiments, the balanced salt solution includes a compounded electrolyte intraocular irrigating solution or a balanced salt intraocular irrigating solution.

The present disclosure also provides a method of preparing the perfusate described in the above embodiment, including the steps of: dissolving NAM in a balanced salt solution to obtain the perfusate containing NAM for intraocular surgery.

The present disclosure also provides use of NAM or the perfusate described in the above technical embodiment in the preparation of a product having a function selected from the group consisting of (1) to (7):
(1) reduction of corneal endothelial cell damage induced by phacoemulsification;
(2) reduction of corneal edema induced by phacoemulsification;
(3) avoiding significant decrease in number of CECs induced by phacoemulsification;
(4) promoting restoration of normal corneal transparency after phacoemulsification;
(5) promoting recovery of normal corneal thickness after phacoemulsification;
(6) maintenance of normal density and normal cellular morphology of CECs, and regular expression and normal distribution of functional proteins after phacoemulsification; and
(7) avoiding a complication arising from phacoemulsification.

In some embodiments, the complication includes ocular hypertension and/or endophthalmitis.

The present disclosure provides use of NAM in the preparation of a perfusate for intraocular surgery. The perfusate prepared from NAM may be applied to phacoemulsification with good results and low cost. In the present disclosure, to address the clinical bottleneck of significant decrease in the number of CECs, corneal edema, and slow recovery of transparency caused by phacoemulsification, NAM is added to the surgical perfusate for anterior chamber, which is applied to phacoemulsification, and the protective effects is observed after surgery based on the morphology and density of CECs, as well as on the thickness and transparency of the cornea. NAM may protect CECs during phacoemulsification in patients with senile cataract (including hypermature stage), diabetic cataract, cataract with corneal endothelial decompensation, and cataract in the period of corneal endothelial function compensation, avoiding a significant decrease in the number of CECs, decreasing the postoperative complications after phacoemulsification in the patients, and promoting the rapid restoration of corneal transparency, which provides an effective strategy for the treatment to realize patient's normal visual function. The results of cellular tests show that NAM treatment could inhibit cell apoptosis of corneal endothelial cell line B4G12 induced by phacoemulsification and promote cell survival; the results of animal experiments show that compared with the intraocular perfusate for anterior chamber alone, the perfusate with the addition of NAM may alleviate the corneal edema induced by phacoemulsification in rabbits, inhibit the increase in the size of the CECs, the cell heteromorphism, and the abnormal expression of the functional proteins ZO1 and ATP1A1, and facilitate the recovery of corneal transparency. The NAM-containing perfusate, when used in phacoemulsification, does not cause complications such as ocular hypertension and endophthalmitis in New Zealand white rabbits, as compared with the control.

### BRIEF DESCRIPTION OF THE DRAWINGS

In order to more clearly illustrate the technical solutions in the embodiments or prior art in the present disclosure, the accompanying drawings to be used in the examples will be briefly described below, and it will be obvious that the accompanying drawings in the following description are only some of the embodiments of the present disclosure, and that for the person of ordinary skill in the field, other accompanying drawings can be obtained based on these drawings without an inventive step.

FIG. 1 is a photograph of gross rabbit corneas 1 day after phacoemulsification according to the present disclosure;

FIG. 2 is an image showing the results of alizarin red staining of rabbit CECs 7 days after phacoemulsification according to the present disclosure;

FIG. 3 is a graph showing the gross rabbit corneas subjected to phacoemulsification with or without NAM addition 1 to 5 days post-operation according to the present disclosure;

FIG. 4 is a chart showing the trend of intraocular pressure (IOP) in rabbits within 7 days after the phacoemulsification with or without NAM addition according to the present disclosure;

FIG. 5 is a chart showing the trend of corneal thickness of rabbit corneas within 7 days after phacoemulsification with or without NAM addition according to the present disclosure;

FIG. 6 is an image showing the staining of functional proteins ZO1 and ATP1A1 in rabbit CECs 7 days after phacoemulsification with or without NAM addition according to the present disclosure; ZO1 is shown in red, ATP1A1 is shown in green, and DAPI is shown in blue;

FIG. 7 a graph showing the effects of NAM and glutamine on CECs in phacoemulsification according to the present disclosure;

FIG. 8 is an image showing the morphology of B4G12 CECs at 1 hour versus 24 hours after phacoemulsification with different concentrations of NAM addition according to the present disclosure.

### DETAILED DESCRIPTION OF THE EMBODIMENTS

The present disclosure provides use of NAM in the preparation of a perfusate for intraocular surgery.

In the present disclosure, the intraocular surgery preferably includes phacoemulsification for cataract and/or vitrectomy. In the present disclosure, the cataract is preferably selected from the group consisting of senile cataract, diabetic cataract, cataract in period of corneal endothelial compensation, and cataract with corneal endothelial decompensation. In the context of the present disclosure, the senile cataract preferably includes a hypermature senile cataract. NAM is the amide form of vitamin B3 and acts as a precursor to NAM adenine dinucleotide (NAD+), which is involved in a variety of cellular biological processes including cellular metabolism, activation of autophagy, anti-oxidative stress, anti-inflammatory responses, and immune responses to physiological or pathological signals, improves cell survival and has a strong cytoprotective effect. It is found in the present disclosure that NAM can be used in the preparation of intraocular perfusates for phacoemulsification and/or vitrectomy.

The present disclosure also provides a perfusate containing NAM for intraocular surgery, the perfusate including NAM and a balanced salt solution. In the present disclosure, the perfusate has a concentration of NAM preferably from 1 to 5 mM, more preferably 2.5 mM. In the present disclosure, the balanced salt solution preferably includes: compound electrolyte intraocular irrigating solution or balanced salt intraocular irrigating solution, more preferably compound electrolyte intraocular irrigating solution. In the present disclosure, the source of the balanced salt solution is not specifically limit, and it is sufficient to use a conventional commercially available product, such as compound electrolyte intraocular irrigating solution (Shike®) or balanced salt intraocular irrigating solution (BSS PLUS®).

The perfusate for intraocular surgery described herein is capable of inhibiting apoptosis of CECs induced by phacoemulsification; reducing corneal edema induced by phacoemulsification; avoiding a significant decrease in the number of CECs induced by phacoemulsification; promoting a rapid restoration of the transparency of the cornea after phacoemulsification; promoting the restoration of the normal thickness of the cornea after phacoemulsification; maintaining a normal density and normal cellular morphology of CECs, and regular expression and normal distribution of functional proteins after phacoemulsification; and avoiding complications (ocular hypertension and/or endophthalmitis) caused by phacoemulsification.

The present disclosure also provides a method for preparing the perfusate described in the above technical solution, including the steps of: dissolving NAM in a balanced salt solution to obtain the perfusate containing NAM for intraocular surgery.

The present disclosure also provides use of NAM or the perfusate described in the above embodiment in the preparation of a product having a function selected from the group consisting of (1) to (7):
(1) suppression of corneal endothelial cell damage induced by phacoemulsification;
(2) reduction of corneal edema induced by phacoemulsification;
(3) avoiding significant decrease in number of CECs induced by phacoemulsification;
(4) promoting restoration of normal corneal transparency after phacoemulsification;
(5) promoting recovery of normal corneal thickness after phacoemulsification;
(6) maintenance of normal density and normal cellular morphology of CECs, and regular expression and normal distribution of functional proteins after phacoemulsification; and
(7) avoiding a complication arising from phacoemulsification. In the present disclosure, the complication includes ocular hypertension and/or endophthalmitis.

In the present disclosure, the product preferably includes a medicament. It is shown in the test that NAM treatment can inhibit apoptosis and promote cell survival of cells subjected to phacoemulsification in a concentration-dependent manner. The perfusate containing NAM for intraocular surgery may protect the transparency and normal thickness of the cornea subjected to phacoemulsification, maintain the normal density and cellular morphology of the CECs, and the regular expression and normal distribution of the functional proteins, and not obviously cause side effects such as ocular hypertension and endophthalmitis.

In the present disclosure, there are no special limitations on the method for using the perfusate, and it is sufficient to adopt the conventional method for using a perfusate in phacoemulsification known to those skilled in the art, for example, compound electrolyte intraocular irrigating solution (Shike®).

In order to further illustrate the present disclosure, the perfusate containing NAM for intraocular surgery, the method of preparation and use thereof according to the present disclosure are described in detail below in connection with the accompanying drawings and examples, but they are not to be construed as a limitation of the protection scope of the present disclosure.

### Example 1

The compound electrolyte intraocular irrigating solution (Shike®, Shenyang Xingqi Pharmaceutical Co., Ltd.) was prepared according to the instructions of the compound electrolyte intraocular irrigating solution (compound electrolyte intraocular irrigating solution Part I: 480 ml of aseptic solution, the main ingredients of which include sodium chloride, potassium chloride, magnesium sulfate, sodium bicarbonate; Part II: 20 ml of sterile solution, the main ingredients of which include dextrose and calcium chloride), and then the Part II solution was transferred into the Part I solution by aseptic injection syringe, a resulting mixture was shaken gently to make the two mix well, and the compound electrolyte intraocular irrigating solution was obtained. Then NAM was added until the concentration of NAM in the perfusate was 1 to 5 mM to obtain the perfusate containing NAM for intraocular surgery.

### Example 2

Materials and methods including the animal level validation versus cellular level validation were described below.
(i) Animal experiment: NAM-containing perfusate protecting rabbit CECs from damage induced by phacoemulsification.

Animals: New Zealand white rabbits (age, 1.5 years; weight, 5-7 kg) were purchased from Jinan Xilingjiao Biological Co., Ltd. New Zealand white rabbits were kept in a pathogen-free environment in the animal house of the Eye Institute of Shandong First Medical University.

Animal model: the pupils were dilated three times with 0.5% compound tropicamide eye drops. Sedation was performed first, and the rabbits were anesthetized according to 20 mg/kg intramuscular injection of xylazine hydrochloride injection and 3% pentobarbital sodium injection (15 mg/kg) intravenously at the ear margin. The right eye of each rabbit was subjected to phacoemulsification for lens removal using the WHITESTAR Signature® Phacoemulsification System (Signature, AMO, USA). The left eye was used as the normal eye. A 3.0-mm clear corneal tunnel incision was made at the position of 11 to 12 o'clock, and continuous curvilinear capsulorhexis was performed in the center of the anterior capsular membrane of the lens, with a torn capsule diameter of approximately 5.0 mm. Phacoemulsification was then performed to suction out the lens contents, including the nucleus of the lens and the residual lens cortex, with the setting of ultrasonication energy of 30-50% and the ultrasonication time of 4-6 min. At the end of the procedure, the New Zealand white rabbits were administered with tobramycin and dexamethasone eye ointment in the conjunctival sac.

Perfusate: balanced salt solution containing 1 mM NAM (refers to compound electrolyte intraocular irrigating solution, Shike®, Shenyang Xingqi Pharmaceutical Co., Ltd.): NAM was added to the balanced salt solution until the concentration of NAM in the perfusate was 1 mM; balanced salt solution containing 2.5 mM NAM: NAM was added to the balanced salt solution until the concentration of NAM in the perfusate was 2.5 mM; balanced salt solution containing 5 mM NAM: NAM was added to the balanced salt solution until the concentration of NAM in the perfusate was 5 mM; balanced salt solution alone as control group: an equal amount of PBS was added to the balanced salt solution.

Immunofluorescence staining: immunofluorescence detection of the expression of functional proteins ZO1 and ATP1A1.

Alizarin Red Staining: gross corneas of New Zealand rabbits were collected and fixed in sterile saline. The cornea was flattened by cutting it into four flaps using ophthalmic microscopic scissors. The corneas were stained with alizarin red staining solution for 2 min, followed by washing the corneas at least three times with sterile saline. Cover slips were put on, changes in the CECs of New Zealand rabbits were observed under a microscope, image acquisition was performed, and all experiments were repeated at least 3 times. Endothelial cell density, coefficient of variation, and hexagonal ratio were determined using alizarin red-stained images of New Zealand rabbit corneas. By using an endothelial microscope by 2 uninformed observers, cell density was normalized to the area of each image in square millimeters. For each image, an area containing at least 60 cells was selected.

Slit lamp photography: changes in corneal transparency in New Zealand rabbits were observed and recorded using a slit lamp.

Corneal thickness test: corneal thickness was analyzed and measured using optical coherence tomography (OCT) to measure central corneal thickness, with no less than three New Zealand rabbits measured at each time point.

Rabbit corneal endothelial cell injury induced by phacoemulsification:

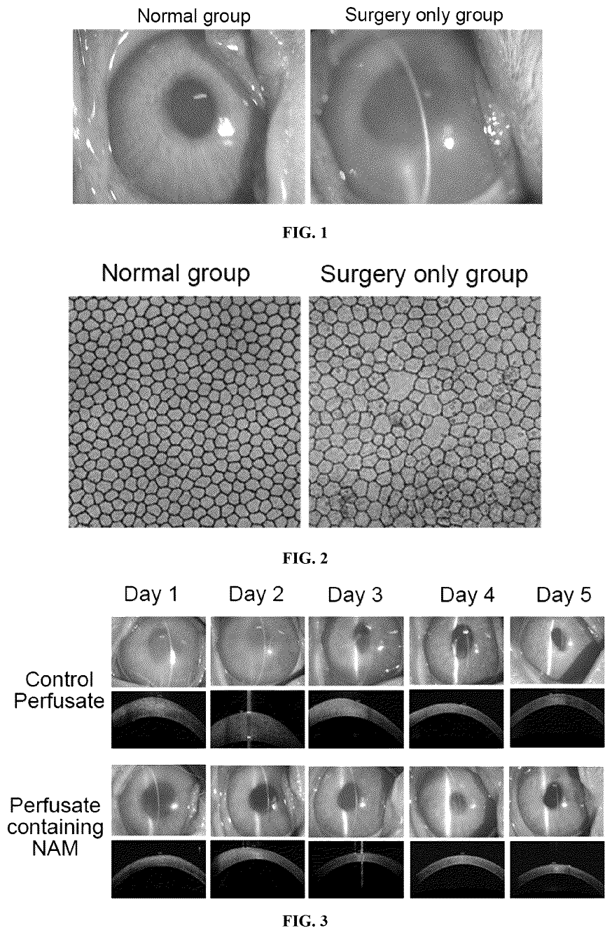

Phacoemusification was performed on New Zealand white rabbits. Compared with normal rabbits, rabbits underwent the surgery exhibited corneal edema and thickening 1 day after the procedure (FIG. 1, photograph of the gross cornea 1 day after phacoemulsification). At the same time, corneal tissues 7 days after phacoemulsification were collected for corneal endothelial stretched preparation and alizarin red staining. Unlike the dense and regular corneal endothelial cell morphology of normal rabbits, the CECs of rabbits underwent phacoemulsification had an enlarged morphology, increased heteromorphism, and a decreased cell density (FIG. 2, alizarin red staining of the CECs 7 days after phacoemulsification).

NAM-containing perfusate protecting the rabbit corneal endothelial cell injury induced by phacoemulsification:

Phacoemulsification was performed on New Zealand white rabbits, where NAM-containing intraocular perfusate was used for irrigation, and perfusate of the balanced salt solution alone was used as a control. Corneal edema, decreased transparency, and increased corneal thickness were observed 1 day after the surgery using perfusate of the balanced salt solution alone, and corneal transparency and thickness gradually recovered to normal over a 5-day period; however, in the NAM-containing intraocular perfusate group, rabbit corneas were protected in terms of transparency and thickness day I after the surgery, and continued to be transparent and maintained normal thickness for 5 days after the surgery (FIG. 3, a graph showing the gross rabbit corneas subjected to phacoemulsification with or without NAM addition 1 to 5 days post-operation).

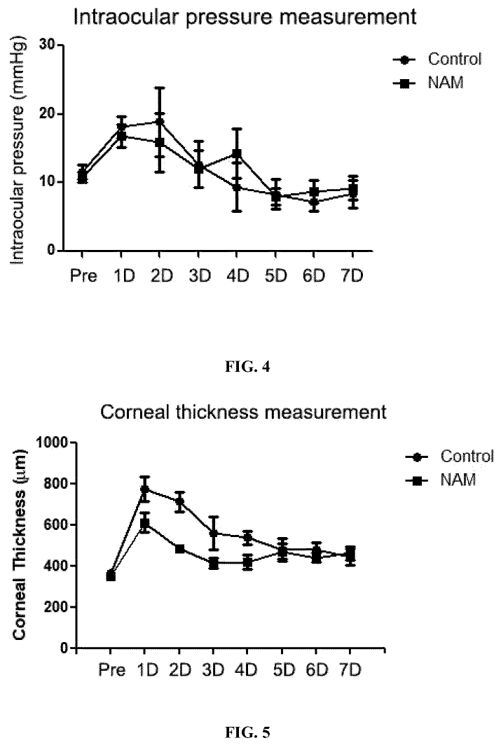

A comparative analysis of the IOP in postoperative rabbit cornea in the control group and the group treated with NAM-containing perfusate revealed that NAM treatment did not cause a pathological increase in IOP within 7 days after surgery, and the trend of IOP was consistent between the two groups when compared with the control group. It was shown that phacoemulsification caused a transient increase in IOP for 3 days, which then basically returned to the baseline level, the elevation of IOP not exceeding the normal range (FIG. 4, a chart showing the IOP profile in rabbits within 7 days after the phacoemulsification with or without NAM addition). Meanwhile, regarding the increased corneal thickness in the control group, the corneal thickness of the rabbits treated with NAM-containing perfusate was lower than that of the control group on the first day after the surgery, and the corneal thickness basically returned to the normal level after 3 days, with a faster rate of recovery of the corneal thickness than that of the control group (FIG. 5, a chart showing the profile of corneal thickness of rabbit corneas within 7 days after phacoemulsification with or without NAM addition).

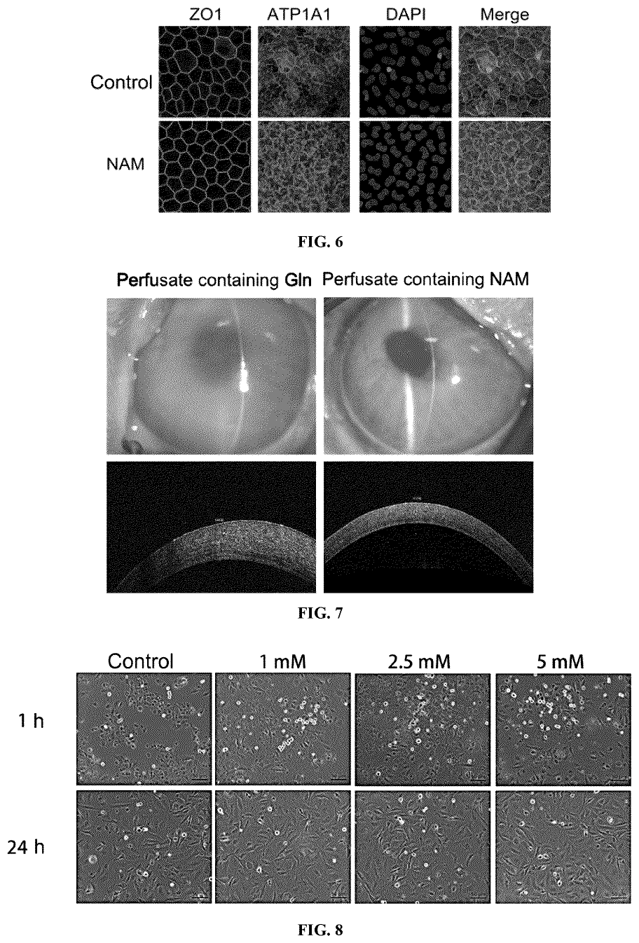

Meanwhile, corneal tissues from both groups were collected for stretched preparation and staining of corneal endothelial cell at 7 days postoperatively. In the control group, enlarged cells with increased heteromorphism, as indicated by ZO1 protein for corneal endothelial cell barrier function, and disturbed expression and localization of the pump function protein ATP1A1 were observed. However, ZO1 staining of rabbit CECs in the NAM-containing perfusate-treated group showed regular cell morphology and normal expression and distribution of ATP1A1 (FIG. 6, an image showing the staining of functional proteins ZO1 and ATP1A1 in rabbit CECs 7 days after phacoemulsification with or without NAM addition; ZO1 is shown in red, ATP1A1 is shown in green, and DAPI is shown in blue).

In addition, glutamine (Gln) has been reported in the literature to have a protective effect on CECs in mice treated with high IOP. In the present disclosure, Gln was added to the perfusate as a control, which was applied to a rabbit lens phacoemulsification model. FIG. 7 is a graph showing the effects of NAM and Gln on CECs in phacoemulsification according to the present disclosure. It was shown that at 2 days after surgery, the rabbit corneas in the NAM-treated group were transparent, while the rabbit corneas in the Gln group were still edematous. The corneal thickness recovered quickly in the NAM-treated group, while that in the Gln group recovered more slowly. The results suggested that NAM has a protective effect on CECs when used in phacoemulsification, while Gln has a poor protective effect on CECs in phacoemulsification although it has a protective effect on mouse CECs during high IOP treatment.

In summary, the results showed that the NAM-containing perfusate was able to promote rapid recovery of corneal transparency and normal thickness in the corneas subjected to phacoemulsification, maintain the normal density and cellular morphology of CECs, and the regular expression and normal distribution of functional proteins, and did not significantly cause side effects such as ocular hypertension and endophthalmitis.
(ii) Cellular assay: NAM inhibiting cultured B4G12 cell damage induced by phacoemulsification in vitro.

Preparation for cell culture in vitro: Human CECs line B4G12 was selected for culture, original medium was discarded, 3 mL of PBS buffer was added, 6.0 cm cell culture dish was shaken gently, rinsed twice, and PBS buffer was discarded. 1.0 mL of trypsin was added to completely cover the bottom of the dish, and the dish was placed in the incubator for 5 min. The dish gently shaken, tapped, and observed with a microscope. The adherent cells became suspended round cells, then 1.5 mL of complete medium [each 50 mL of human corneal endothelial cell basal medium (H-SFM, Creative Bioarray, New York, USA) supplemented with 1.5 mL of FBS and 0.5 mL of penicillin-streptomycin solution] was added to the flask on the laminar flow cabinet, and trypsin digestion of cells was stopped. The cells on all parts of the bottom of the flask were gently pipetted using the tip of the pipettor so that the cells became fully in suspension. The cell suspension was transferred to a 15 mL centrifuge tube and subjected to centrifugation at 1200 rpm for 5 min. After the centrifugation, the supernatant was discarded, 3 mL of complete medium was added to the centrifuge tube, and a resulting mixture was gently pipetted to eveness. The cells were allowed to resuspend sufficiently, then inoculated into 48-well cell culture plates at a cell density of 1×10⁵ and incubated for 24 hours for subsequent experiments.

Experimental grouping: in vitro cell experiments included a control group and experimental treatment groups with different concentrations of NAM. Control group: complete medium was utilized; experimental treatment group: (1) medium containing 1 mM NAM: NAM was added to the complete medium until the concentration of NAM reached 1 mM in the medium; (2) medium containing 2.5 mM NAM: NAM was added to the complete medium until the concentration of NAM reached 2.5 mM in the medium; (3) medium containing 5 mM NAM: NAM was added to the complete medium until the concentration of NAM reached 5 mM in the medium.

Phacoemulsification: the culture medium was aspirated to replace with a balanced salt solution. The Oertli phacoemulsification Instrument (CataRhex Swisstech, Oertli Instrumente AG, Switzerland) was prepared and commissioned. The phacoemulsification tip was inserted into the balanced salt solution of the culture well plate, with an ultrasonic energy of 20-40% and and ultrasonic time of 20-30 seconds, then the balanced salt solution with four groups of culture medium above was replaced, and placed in the incubator and continue to incubate for 1 h, 6 h, 12 h and 24 h, then the cells were observed under the microscope and pictures were taken to record the cell status.

NAM treatment protecting human CECs from damage and apoptosis induced by phacoemulsification:

The human corneal endothelial cell lines were subjected to phacoemulsification with a sonication time of 20 seconds and a sonication energy of 30%. The changes of CECs at 1 hour and 24 hours after phacoemulsification with 1 mM, 2.5 mM and 5 mM NAM were observed. It was shown that NAM treatment could inhibit apoptosis induced by phacoemulsification and promote cell survival in a concentration-dependent manner (FIG. 8, an image showing the morphology change of B4G12 CECs at 1 hour versus 24 hours after phacoemulsification with different concentrations of NAM addition according to the present disclosure).

Although the above examples make an exhaustive description of the present disclosure, it is only a part of the embodiments of the present disclosure, not all of the embodiments. One can obtain other embodiments according to the present embodiments on the premise of not involving an inventive step, and these embodiments all belong to the protection scope of the present disclosure.

1. (canceled)

2. (canceled)

3. (canceled)

4. A perfusate containing nicotinamide (NAM) for intraocular surgery, comprising NAM and a balanced salt solution.

5. The perfusate of claim 4, wherein NAM in the perfusate has a concentration of 1 to 5 mM.

6. The perfusate of claim 4, wherein the balanced salt solution comprises compounded electrolyte intraocular irrigating solution or balanced salt intraocular irrigating solution.

7. A method for preparing the perfusate of claim 4, comprising dissolving NAM in a balanced salt solution to obtain the perfusate containing NAM for intraocular surgery.

8. A method for using the perfusate of claim 4, wherein the perfusate has a function selected from the group consisting of (1) to (7):
(1) suppression of corneal endothelial cell damage induced by phacoemulsification;
(2) reduction of corneal edema induced by phacoemulsification;
(3) avoiding significant decrease in number of corneal endothelial cells (CECs) induced by phacoemulsification;
(4) promoting restoration of normal corneal transparency after phacoemulsification;
(5) promoting recovery of normal corneal thickness after phacoemulsification;
(6) maintenance of normal density and normal cellular morphology of CECs, and regular expression and normal distribution of functional proteins after phacoemulsification; and
(7) avoiding a complication arising from phacoemulsification.

9. The perfusate of claim 8, wherein the complication comprises ocular hypertension and/or endophthalmitis.

10. A method for performing intraocular surgery, comprising administering the perfusate of claim 4 to a subject in need thereof, wherein the perfusate protects CECs during the intraocular surgery.

11. The method of claim 7, wherein the NAM in the perfusate has a concentration of 1 to 5 mM.

12. The method of claim 7, wherein the balanced salt solution comprises compounded electrolyte intraocular irrigating solution or balanced salt intraocular irrigating solution.

13. The perfusate of claim 8, wherein the NAM in the perfusate has a concentration of 1 to 5 mM.

14. The perfusate of claim 8, wherein the balanced salt solution comprises compounded electrolyte intraocular irrigating solution or balanced salt intraocular irrigating solution.

15. The method of claim 10, wherein the NAM in the perfusate has a concentration of 1 to 5 mM.

16. The method of claim 10, wherein the balanced salt solution comprises compounded electrolyte intraocular irrigating solution or balanced salt intraocular irrigating solution.

17. The method of claim 10, wherein the intraocular surgery comprises phacoemulsification for cataract and/or vitrectomy.

18. The method of claim 17, wherein the cataract is selected from the group consisting of senile cataract, diabetic cataract, cataract in period of corneal endothelial compensation, and cataract with corneal endothelial decompensation.
